(19) United States
(12) Patent Application Publication
(10) Pub. No.: US 2010/0196507 A1
(43) Pub. Date: Aug. 5, 2010

(54) Treatment of Wounds and Compositions Employed

(75) Inventors: Stephen H. MONROE, Memphis, TN (US); Matthias Johannes HOEKSTRA, Amsterdam (NL)

Correspondence Address: SPENCER FANE BRITT & BROWNE LLP, 1 NORTH BRENTWOOD BLVD., SUITE 1000, ST. LOUIS, MO, 63105-3925, US

(73) Assignee: GREYSTONE MEDICAL GROUP, INC., Fort Myers, FL (US)

(21) Appl. No.: 12/565,244

(22) Filed: Sep. 23, 2009

Related U.S. Application Data
(63) Continuation of application No. 11/779,026, filed on Jul. 17, 2007, now abandoned.
Application No. 11/779,026 is a continuation of application No. 11/523,869, filed on Sep. 20, 2006, now abandoned.
Application No. 11/523,869 is a continuation of application No. 11/081,119, filed on Mar. 15, 2005, now abandoned.
Application No. 11/081,119 is a continuation of application No. 10/305,713, filed on Nov. 27, 2002, now abandoned.
(60) Provisional application No. 60/334,337, filed on Nov. 29, 2001.

(51) Int. Cl. A61K 33/30; A61P 17/02

(52) U.S. Cl. 424/642

(57) ABSTRACT

A synthesized composition containing zinc ions, calcium ions, rubidium ions and/or potassium ions in a pharmaceutically acceptable carrier, which, when applied to an open wound, effectively modulates the activity of at least MMP-2 and/or MMP-9 in the wound. A method for treatment of wounds is disclosed.

### RELATED APPLICATIONS

This application is a non-provisional application claiming priority based on Provisional application Ser. No. 60/334,337, filed Nov. 29, 2001, said Provisional application, in its entirety, being incorporated herein by reference.

### FIELD OF INVENTION

This invention relates to the treatment of wounds, particularly open wounds which resist healing, such as decubitus ulcers and diabetic ulcers. It further relates to the use of inorganics as an aid in the establishment and/or control over the chemical environment associated with extra cellular matrices.

More particularly, this application relates to synthetic compositions for the modulation of matrix metalloproteinases (MMPs), especially in the treatment of open chronic wounds and other skin disorders.

In the prior art it is known that there exist within the human body a plurality of metal metalloproteinases. It has been suggested that at least certain of these MMPs lie relatively dormant (“Pre-MMP”) until activated, whereupon various of the MMPs affect cellular growth or lack of growth, the MMPs acting at least in part through the extracellular matrix (ECM) of the cells

MMP-2 has been particularly indicated in the healing of wounds. In its inactive state, Pro-MMP-2 includes a ribbon of protein which covers its active site. Removal (cleavage) of this protein must occur before this MMP can become activated. This has been termed a “Cysteine switch”. Zinc ions at the active site have been noted to activate MMP-2. Also, calcium ions at a secondary site are believed to provide the MMP with the proper geometry in its active state. Inhibitors of metalloproteinase (TIMP) have been identified.

### SUMMARY OF THE INVENTION

The present inventors have identified MMP-2 and MMP-9 in increased quantities both in the peripheral region and particularly within the deep recesses of a chronic wound. It has also been a noted increase in these MMPs in “difficult to heal” open wounds. Further the present inventors have discovered a synthesized composition containing zinc ions, calcium ions, rubidium ions and/or potassium ions in a pharmaceutically acceptable carrier, which, when applied to an open wound, after two weeks of treatment, effectively shuts down the activity of MMP-2 and/or MMP-9 in the wound as evidenced by analysis of wound cultures for the presence of MMPs 2 and 9, and resulting visually observable improvement in the healing of the wound. The visually observable improvement in the healing process of the wound is dramatic and takes place within an unexpectedly short time frame.

Moreover, continued application of the composition of the present invention to a chronic wound site has been found effective in bringing about complete healing of chronic wounds, often within a matter of weeks. Especially, the composition containing the effective ingredients of the present invention has been determined effective to modulate the presence, hence the activity of, MMPs within the deeper inner recesses of the wound, as opposed to the outer peripheral regions of the wound. The present composition further appears to act as an oxygen scavenger and thereby eliminating or materially reducing the ill effects of oxygen radicals within the inner recesses of the wound.

Wounds such as decubitus ulcers, and deep burns have been effectively treated employing the concepts of the present invention.

### BRIEF DESCRIPTION OF DRAWINGS

FIG. 1 is a photograph of depicting a wound having applied thereto a composition embodying the present invention;

FIGS. 2-5 are photographs of typical non-responding wounds;

FIGS. 6 and 7 are photographs of the leg wound of Example I, depicting the wound of Example I before and after treatment, respectively, in accordance with the present invention;

FIG. 8 is a photograph of the leg wound of Example I before treatment in accordance with the present invention;

FIG. 9 is a microphotograph of a biopsy of the wound depicted in FIG. 8;

FIG. 10 is a microphotograph depicting the levels of MMP-2 in the upper layers of Zones A and B of FIG. 9;

FIG. 11 is a microphotograph depicting the levels of MMP-2 in the deeper layers of Zone C of FIG. 9;

FIG. 12 depict the appearance of Zones A, B and C of FIG. 9 after 14 days of treatment in accordance with the present invention;

FIG. 13 is a photograph depicting an external view of the wound depicted in FIG. 8 after 14 days of treatment;

FIG. 14 is a microphotograph of Zone B of FIG. 9 after 14 days of treatment;

FIG. 15 is a photograph of the wound of Example I after 6 weeks of treatment;

FIG. 16 is a microphotograph of a biopsy of the wound depicted in FIG. 15;

FIG. 17 is a photograph of the wound of Example II prior to commencement of treatment in accordance with the present invention;

FIG. 18 is a microphotograph of the wound depicted in FIG. 17 after complete healing of the wound following 4½ weeks of treatment;

FIG. 19 is a photograph of the wound of Example III prior to the start of treatment in accordance with the present invention;

FIG. 20 is a photograph depicting the wound of Example III as being fully healed after 7 weeks of treatment in accordance with the present invention;

FIG. 21 is a photograph of the wound of Example IV prior to the commencement of treatment in accordance with the present invention;

FIG. 22 is a photograph of the wound of Example IV as being fully healed at week 7 following treatment in accordance with the present invention;

FIG. 23 is a microphotograph of the wound of Example IV and depicted in FIG. 21;

FIG. 24 is a microphotograph of the wound of Example IV taken in the superficial region of the wound prior to treatment in accordance with the present invention;

FIG. 25 is a microphotograph of the wound of Example IV taken in the deep regions of the wound prior to treatment in accordance with the present invention;

FIG. 26 is photograph depicting the wound of Example IV after 3 weeks of treatment in accordance with the present invention;

FIG. 27 is a microphotograph of the wound depicted in FIG. 26;

FIGS. 28 and 29 are microphotographs depicting the progress of healing of the wound of Example IV;

FIG. 30 is a photograph of the wound of Example IV after 5 weeks of treatment in accordance with the present invention;

FIG. 31 is a microphotograph of the wound depicted in FIG. 30;

FIGS. 32 and 33 are microphotographs of the wound of Example IV after 5 weeks of treatment in accordance with the present invention and depicting the decrease in MMP expression;

FIG. 34 is a photograph of a right let wound of Example V prior to commencement of treatment in accordance with the present invention;

FIG. 35 is a photograph showing full healing of the right leg wound of Example V after 84 months of treatment in accordance with the present invention;

FIG. 36 is a photograph of a left leg wound of Example V prior to commencement of treatment in accordance with the present invention;

FIG. 37 is a photograph of the left leg wound of Example V after nine months of treatment in accordance with the present invention;

FIG. 38 is a pictorial representation of the wound healing process;

FIG. 39 is a pictorial representation of the balancing of MMPs within a wound;

FIG. 40 is a pictorial representation of ECM generation and degradation in a wound; and,

FIG. 41 is a pictorial representation of collagen formation in a wound.

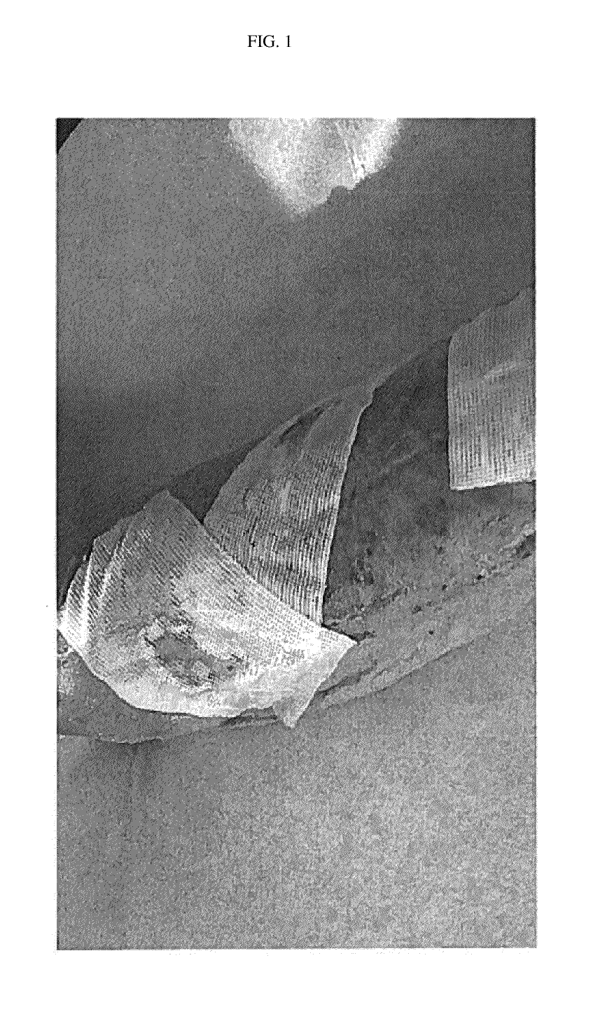

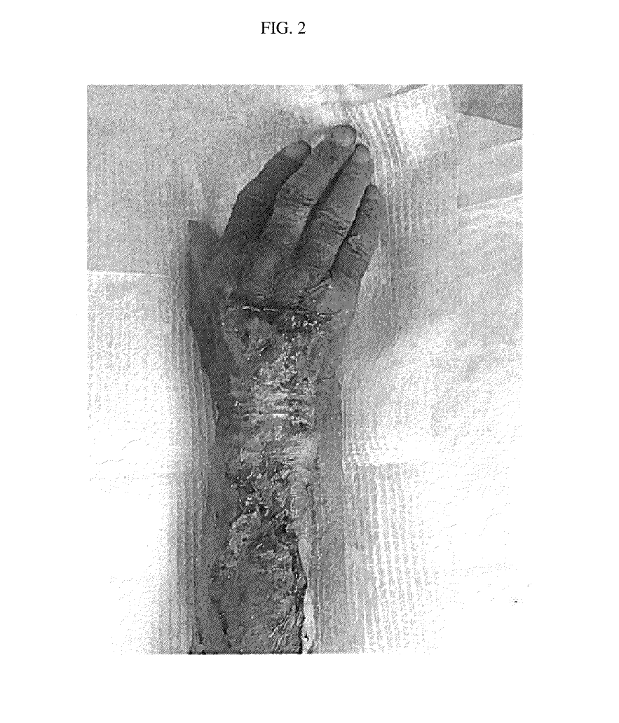

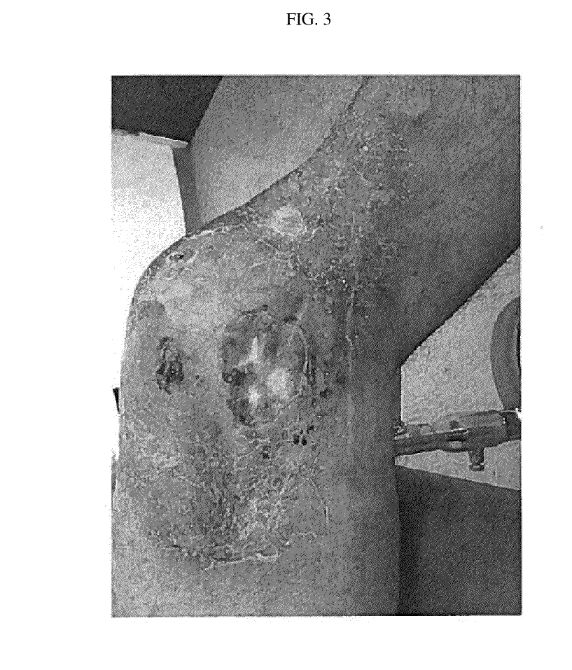

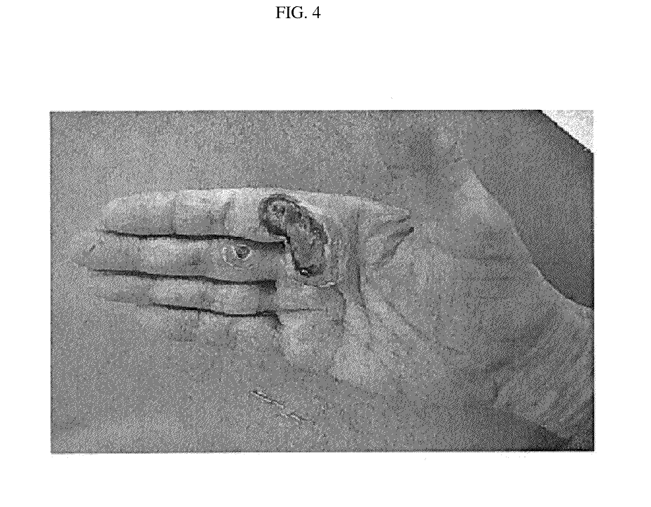

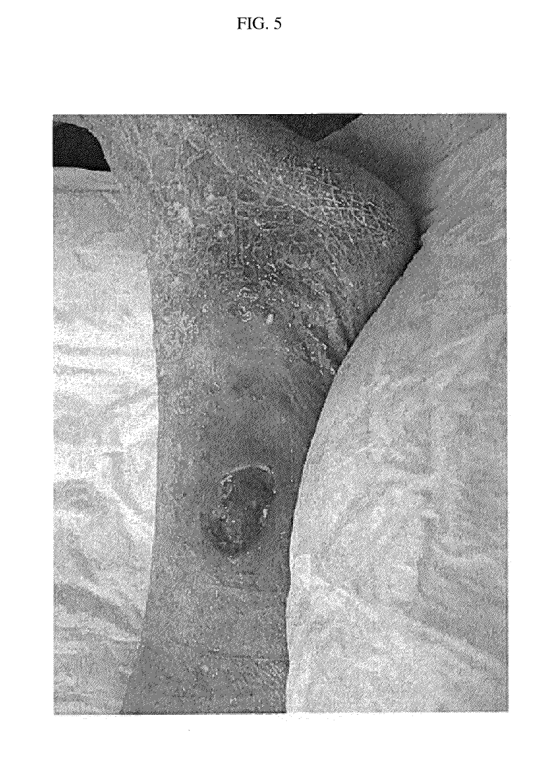

### DETAILED DESCRIPTION OF THE INVENTION

In initial experimentation conducted with rats (partial thickness excision wounds) and Yorkshire pigs (contact burn wounds), the present inventors found that compositions containing the ingredients of the present invention promoted epithelialization, resulting in a more “normal” epidermis. The wound bed contained less activated macrophages, cells staining positive for acid phosphatase.

Infliction of deep, dermal contact wounds in domestic pig models induce defects which are not fully epithelialized, depending on the treatment applied. Tissue biopsy wounds are deep full thickness skin defects measuring 9 by 2 cm. Such biopsy wounds have a slow tendency to epithelialize. When excision biopsy wounds are filled up with granulation tissue there is a clear visible healing of the wound by contraction. These wounds are ideal test models to get a clear macroscopic impression of the efficacy of test substances applied. Compositions containing the ingredients of the present invention have been found to convert such wounds, which mainly healed by epithelialization starting a couple of days after the first application. Also, such biopsy wounds showed clear epithelialization instead of contraction in comparison with wounds treated with the present compositions.

Employing the domestic pig model, compositions containing the ingredients of the present invention were compounded and tested. These tests showed clear expression of MMP-2 in untreated wounds. Only minimal expression of MMP-2 was observed in comparative wounds treated with a composition containing the ingredients of the present invention.

The foregoing tests were followed by in vitro human studies employing a composition containing the ingredients of the present invention. In these tests, the composition was impregnated onto an ethylene vinylacetate carrier for form an impregnated dressing for the wound site.

In the present studies, 31 patients were initially involved in the study. Five patients dropped out of the study and eight patients are receiving continuing treatment. Of these patients, the wound(s) of 18 patients were completely healed with an average healing time of 10 weeks. All of the patients in the study responded positively.

The following specific examples are provided as exemplary of the results observed in the human studies. In each instance, a composition in accordance with the present invention, on an EVOH carrier defining a bandage was applied to the wound site. The bandage was removed at various intervals and replaced with a fresh bandage. A sufficient quantity of the composition of the present invention was placed on the carrier to substantially fully fill the wound cavity.

### Example I

### Female 74 years of age

History:
Rheumatoid Arthritis.

Medication:
High doses of steroids.

Type wound:
Post traumatic ulcer on lateral lower leg after infected hematoma.

Duration of Wound

Wound had existed for more than one year prior to commencement of present treatment.

Earlier Treatments
DUODERM
HYDROGEL
Vacuum system
Honey and SSD,

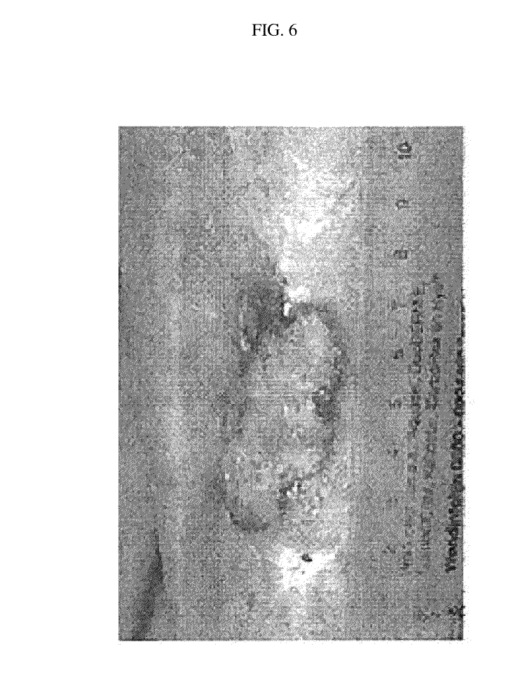

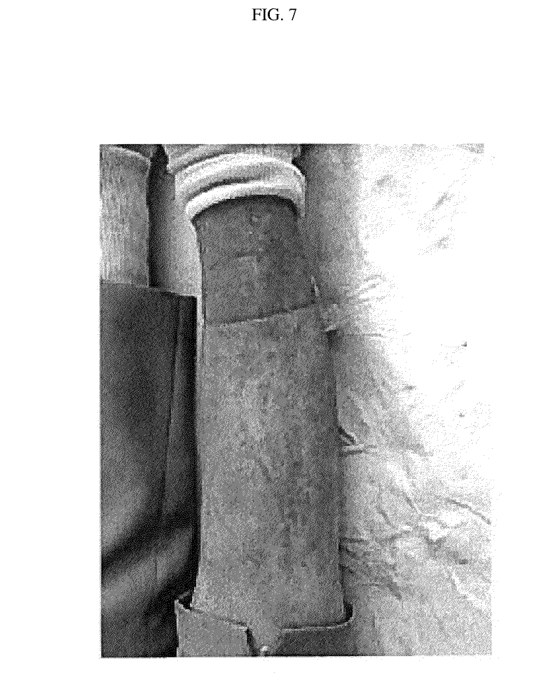

FIG. 6 depicts this wound at the time of commencement of treatment. Prior to entry into the present study. FIG. 7 depicts the healed wound after 30 weeks of treatment. It is noted that after 12 weeks of treatment with the composition, this patient was treated with steroids. This action was noted to delay the healing process and was discontinued. Thus, without the intervention of the steroid treatment, the healing time for this patient would have been shorter.

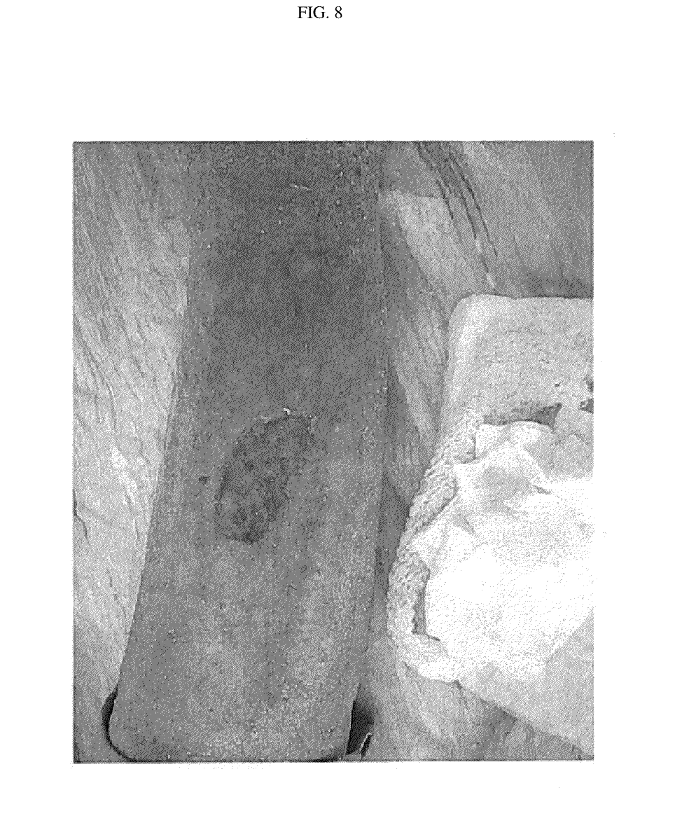

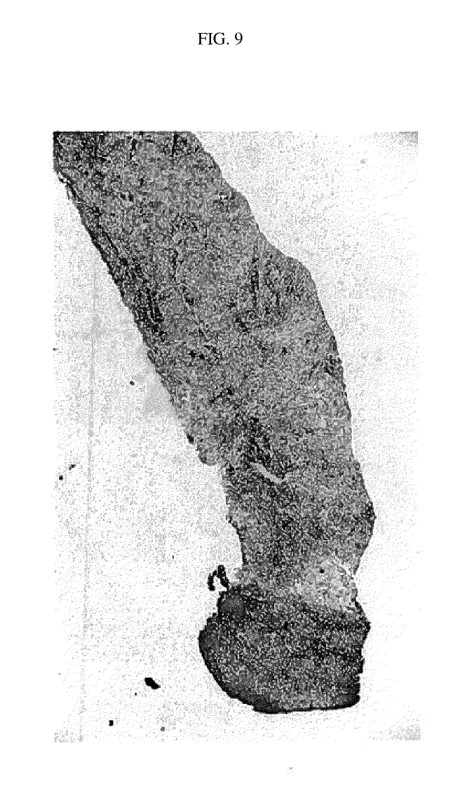

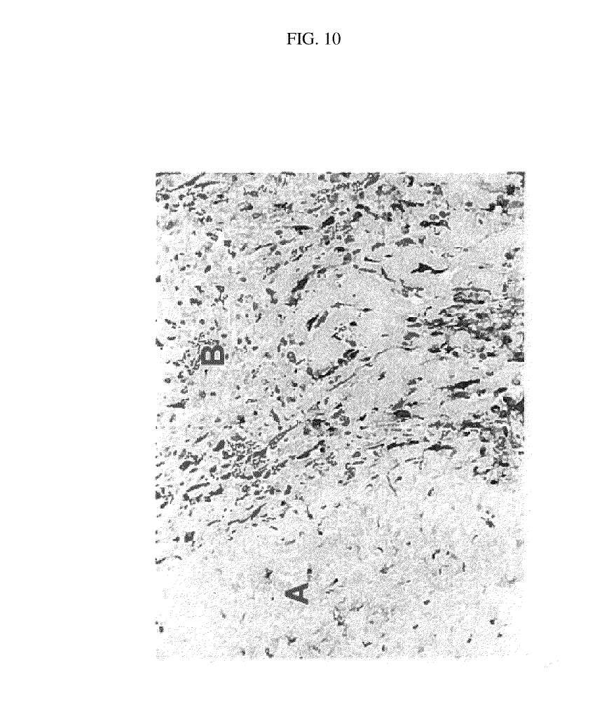

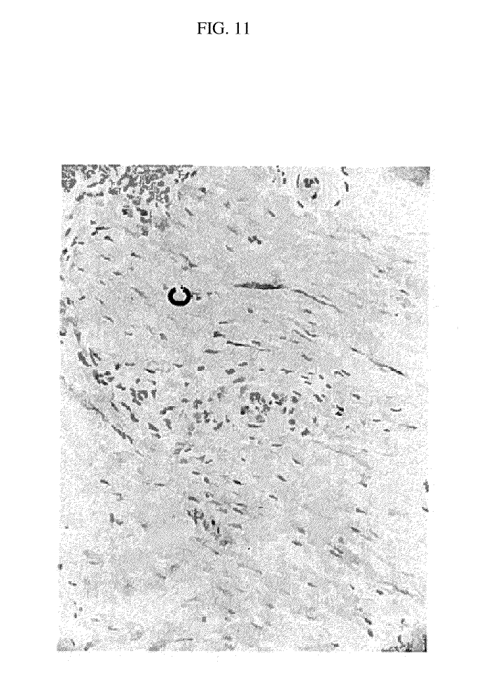

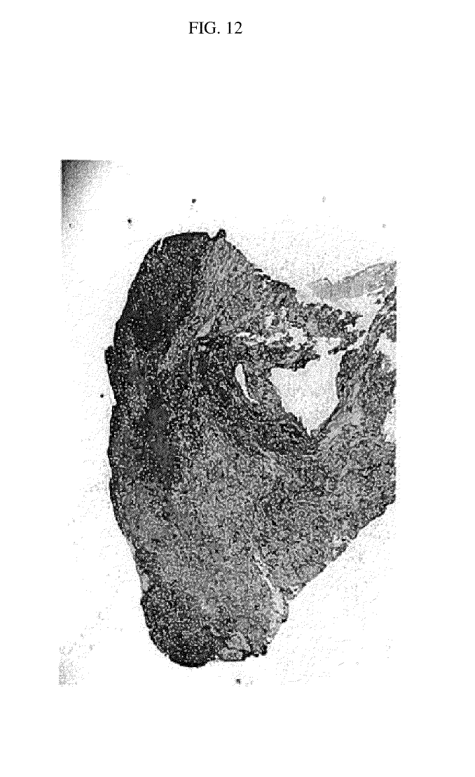

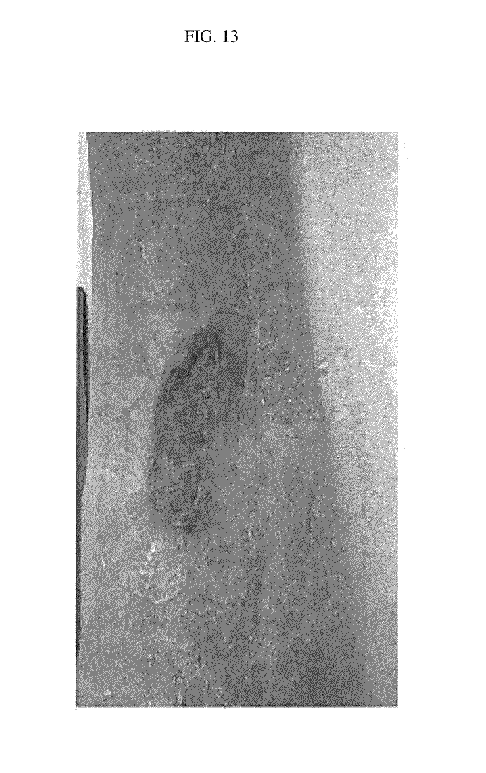

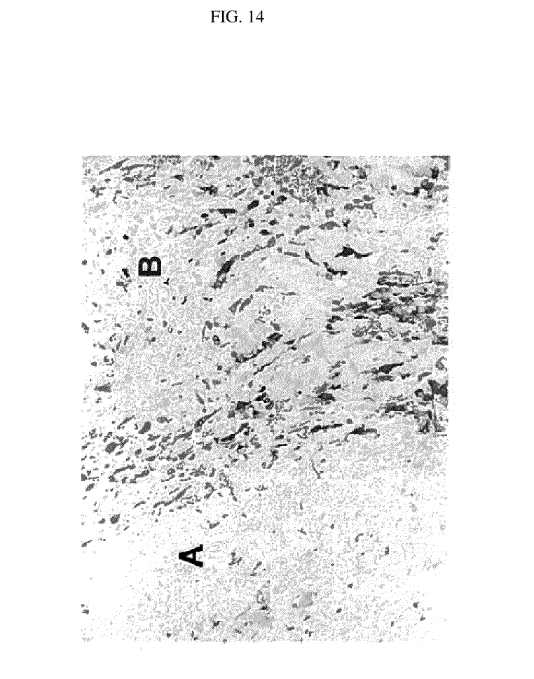

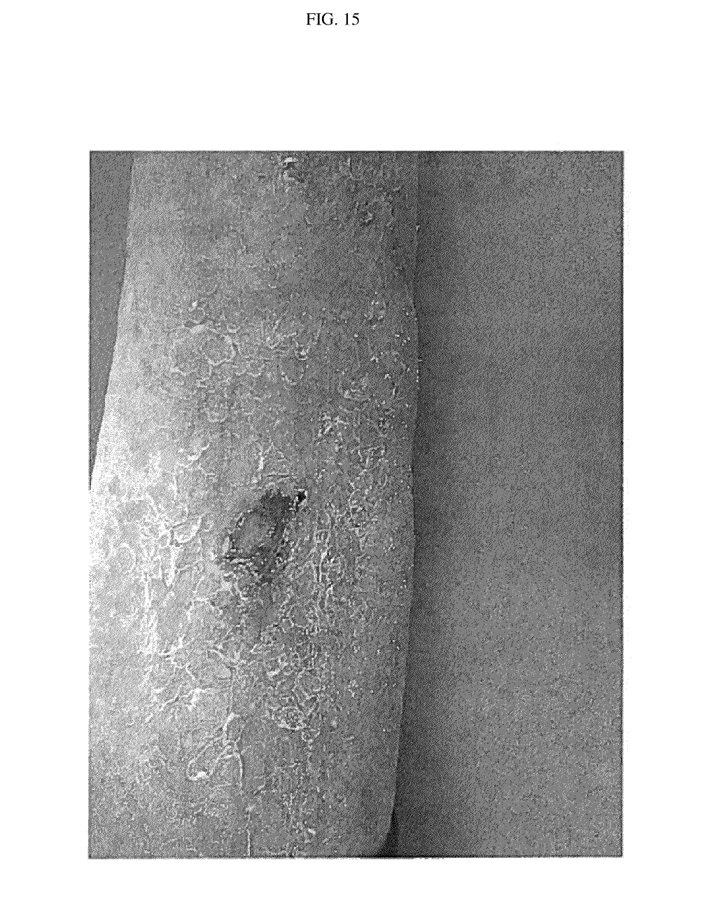

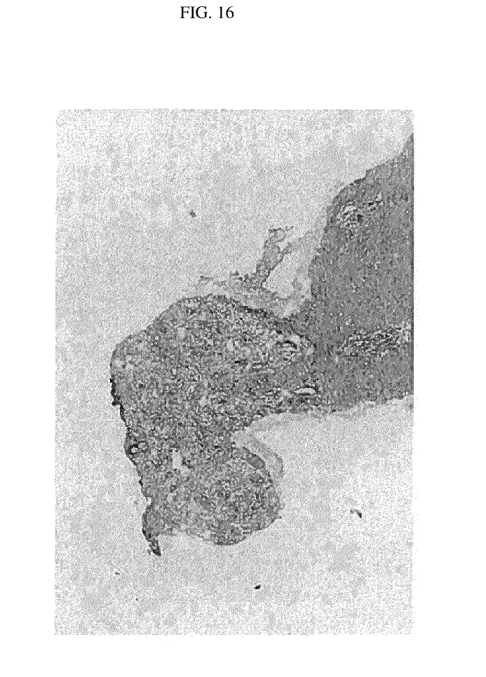

Referring to FIGS. 8 and 9, at Day One, the wound of this patient was about 6 cm long and about 2 cm wide. The wound extended deeply into the leg. A biopsy of the wound is depicted in FIG. 9 wherein a cross-section of the wound is depicted as including Zones A, B and C. Zone A consists of a broad fibrin layer with necrotic cellular debris. Zone B is a rather broad zone with breakdown of matured collagen and inflammation. Zone C is adjacent the bottom of the wound and depicts a decline of inflammation at this location. Examination of the Day One biopsy for MMP-2 prior to the treatment showed fibroblasts in the upper layers of the wound to be expressing high levels of MMP-2 (FIG. 10). This same biopsy depicted no more than a single fibroblast staining positive for MMP-2 in the deeper layers of the wound. As depicted in FIGS. 12 and 13, after 14 days of treatment with the composition, all zones are readily identifiable, with the fibrin cap depicting large accumulations of neutrophils. Zone B at this time of treatment is identifiable directly beneath the fibrin cap and shows less old collagen and the appearing of neo-dermis. FIG. 13 shows the overall appearance of the wound after 14 days treatment and clearly indicates both a “cleaner” wound and reduction in the overall size of the original wound. Biopsies of the wound after 14 days of treatment showed no clear change in the expression of MMP-2 in Zone B (FIG. 14). As shown in FIGS. 15 and 16, after 6 weeks of treatment, the wound was further decreased in size and healing was progressing. A biopsy of the wound at this time showed that the necrotic cap had vanished and the neo-dermis was healthy. Further, the biopsy the expression of MMP-2 within the wound had declined to near zero, coinciding with the healthy appearance of the neo-dermis.

Between the 6^{th} and 12^{th} weeks of treatment of the present patient, steroid treatment was conducted. At week 12, a biopsy of the wound clearly showed that the fibroblasts began again to express MMP-2. Treatment of the wound using steroids was ceased and the wound fully healed within a total treatment time of 30 weeks as shown in FIG. 7.

### Example II

### Male 75 years of age

History
Decompensation cordis
Vascular insufficiency
Diabetes Mellitus

Wound type
Post traumatic
Lacerations

Duration of Wound
Wound had existed for weeks

Earlier Treatments
ADAPTIC
SSD (FLAMMAZINE)

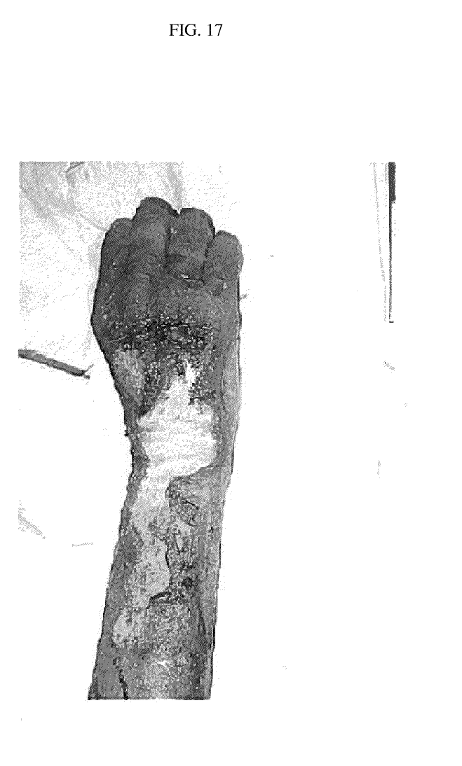

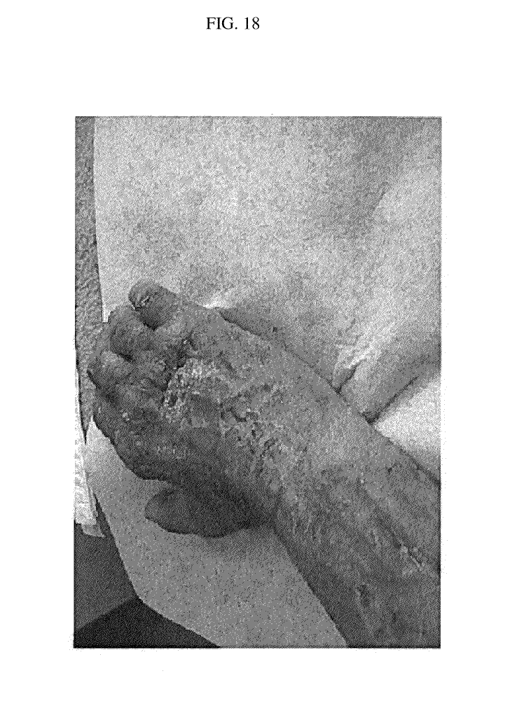

FIG. 17 depicts the wound of Example II at Day One, prior to the commencement of treatment with the composition. FIG. 18 depicts that portion of the arm treated with the composition as being completely healed after 4½ weeks of treatment.

### Example III

### Male 81 years of age

Type of wound
2^{nd} and 3^{rd} degree burns by electricity

Duration of wound
Wound had existed 16 days

Earlier Treatments
SSD (FLAMMAZINE)
ELASTO-GEL

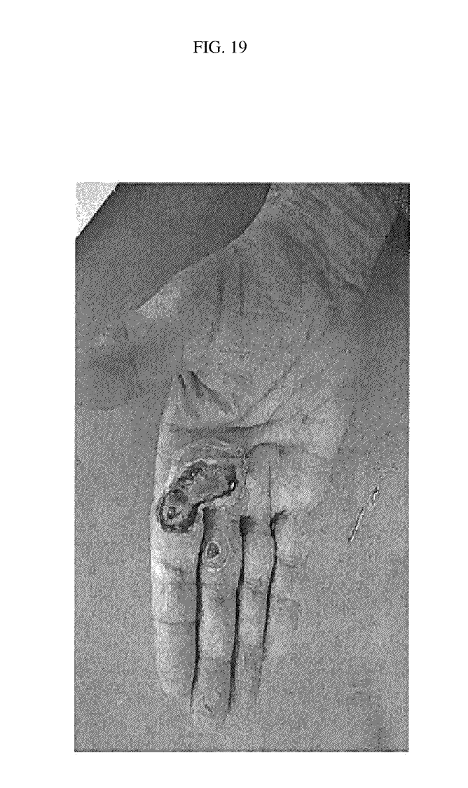

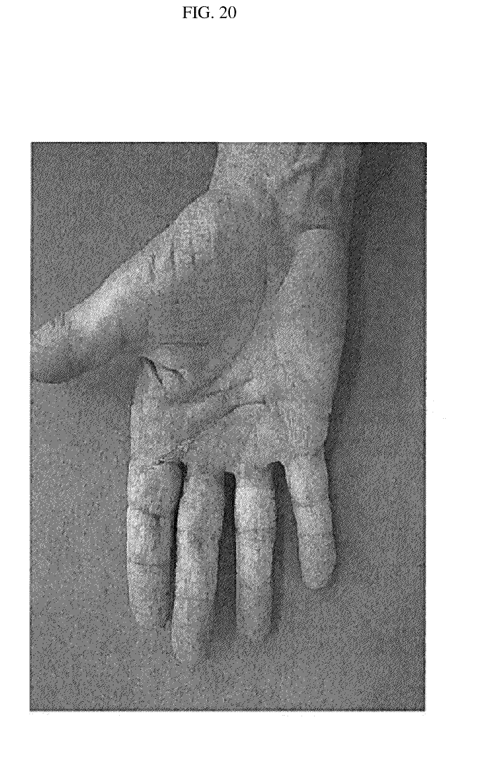

FIG. 19 depicts this wound at Day One of the commencement of treatment of the wound with the composition. FIG. 20 depicts the completely healed wound after 7 weeks of treatment.

### Example IV

### Male 57 years of age

History
Diabetes Mellitus
Pilonfracture
Osteosynthesis

Type of wound
Post traumatic ulcer lateral malleoulus

Duration of wound
Wound had existed for more than one year

Earlier Treatment
KALTOSTAT

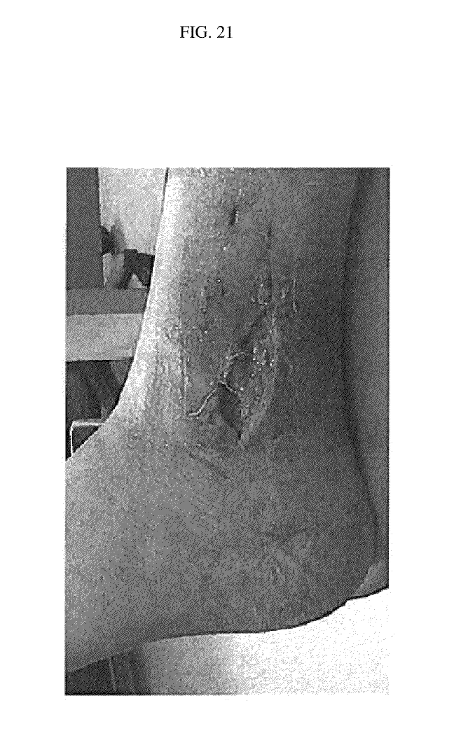

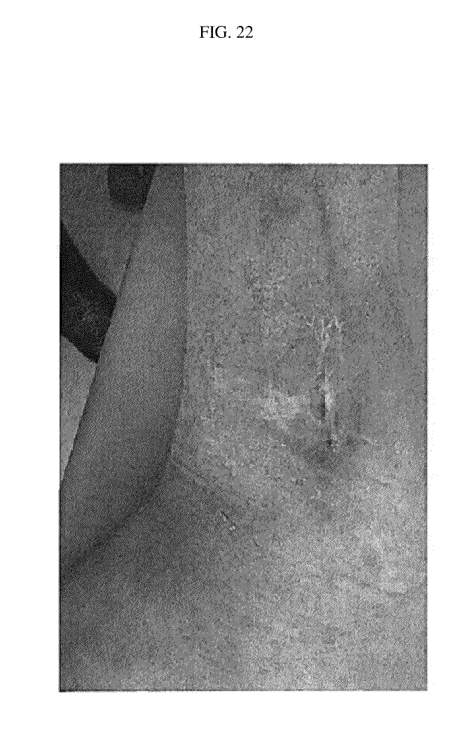

FIG. 21 depicts the present wound at Day One and prior to commencement of treatment. FIG. 22 depicts the completely healed wound at week 7.

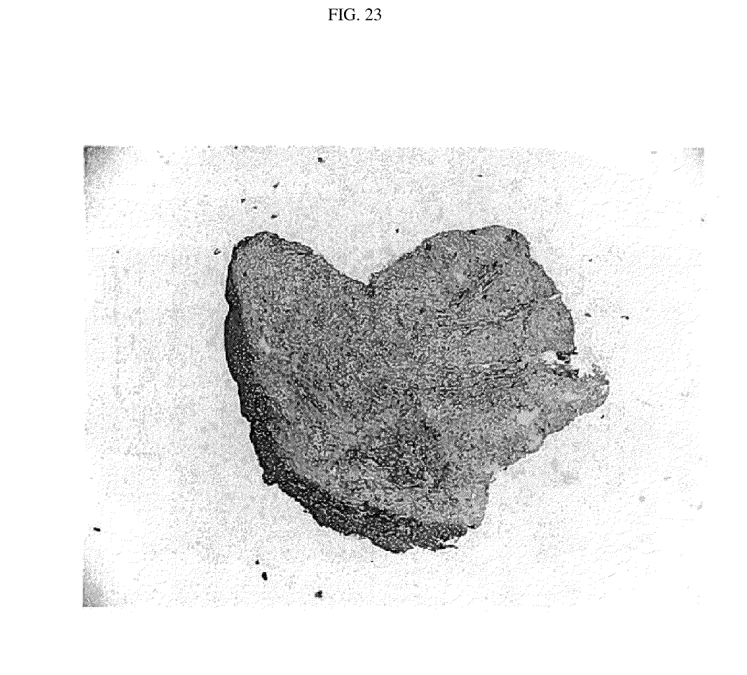

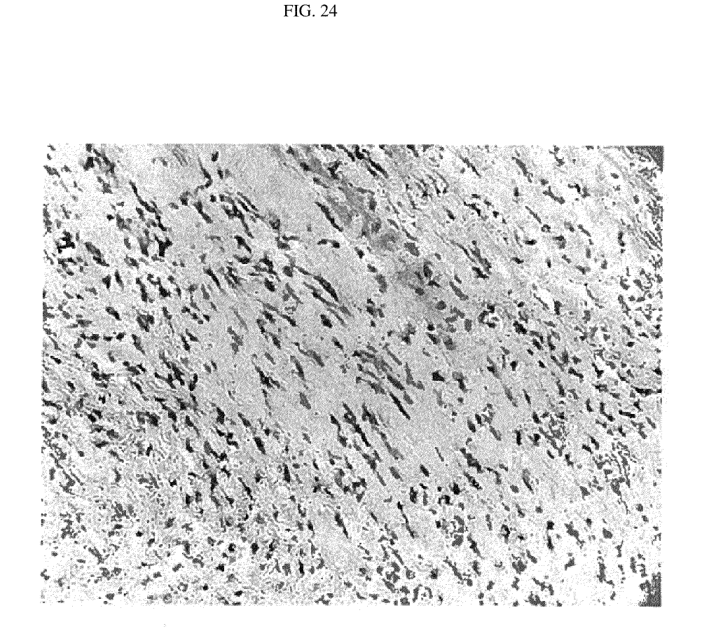

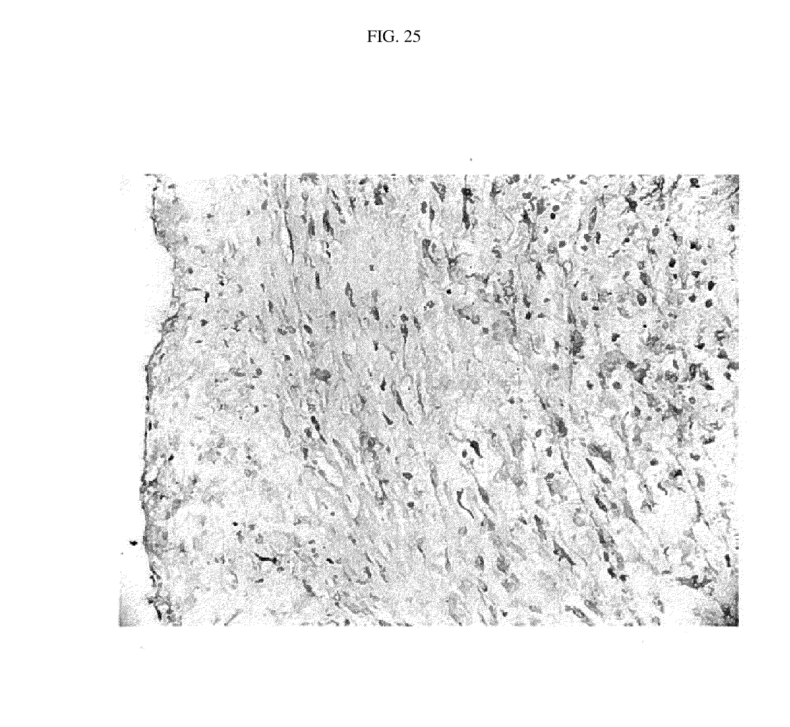

A biopsy of a cross-section of the wound depicted in FIG. 21 is shown in FIG. 23. Of note is the fibrous cap consisting of fibrin and dead cells on top of the wound. FIGS. 24 and 25 show the expression of MMP-2 being high in the superficial and deep regions of the wound, at Day One.

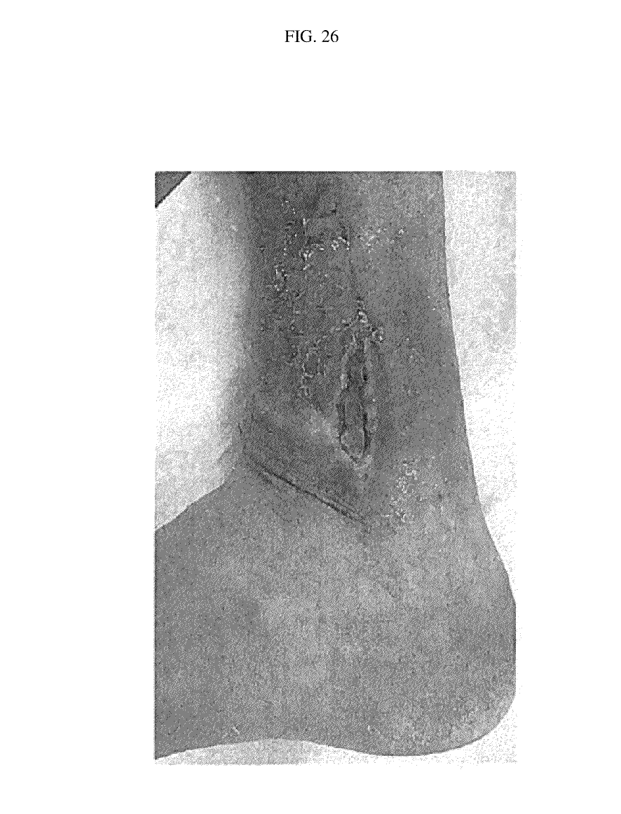

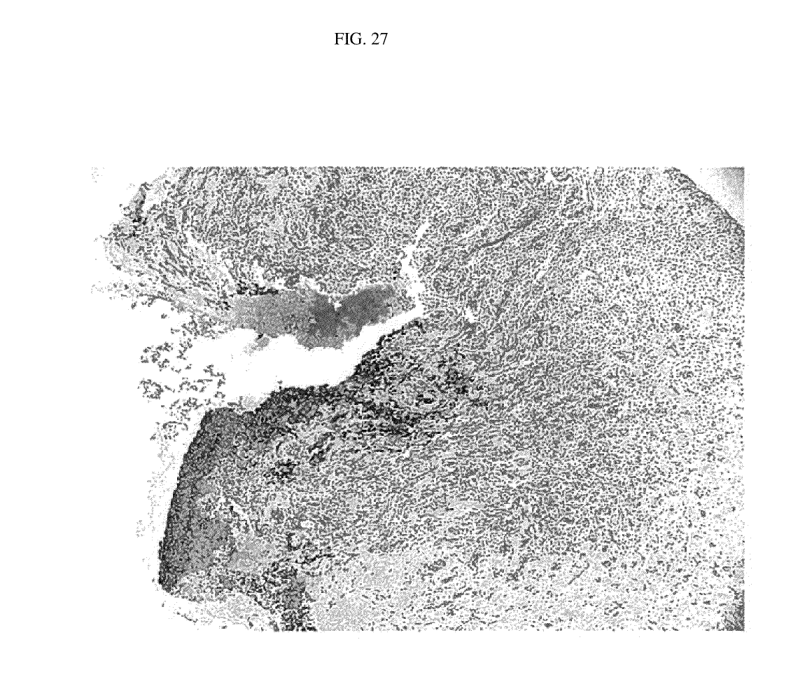

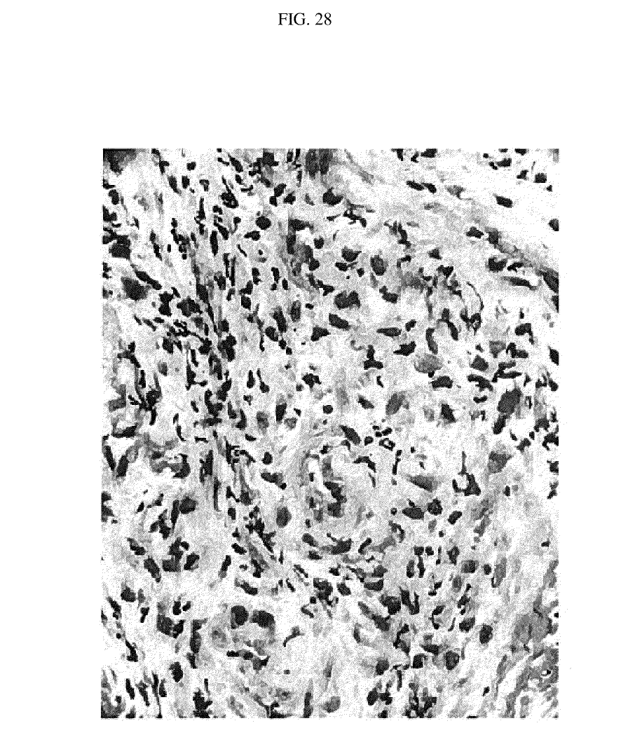

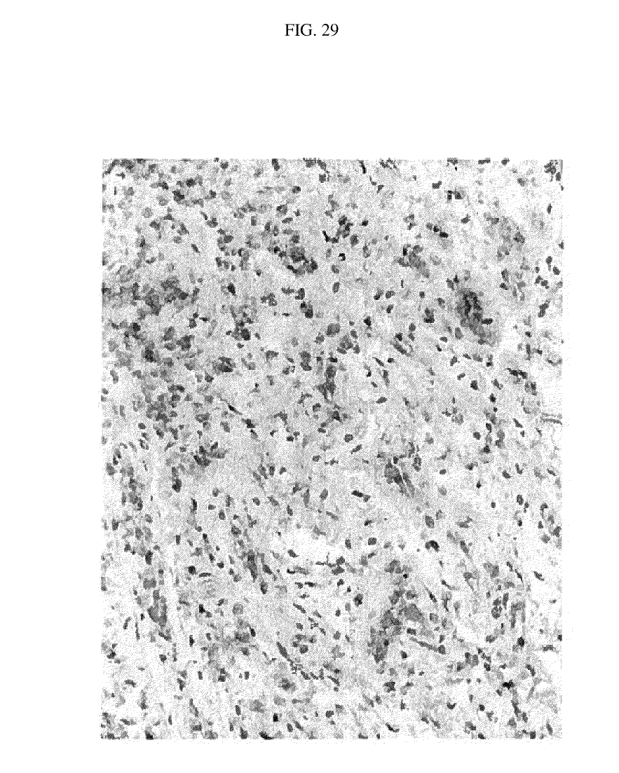

At week 3 of treatment, the wound showed clear progress in epithelial outgrowth (FIG. 26) and reduction in the size of the fibrinous cap (FIG. 27). Examination of biopsies of the wound at week 3 further showed an increase in fibroblast and blood vessels (FIG. 28) and diminished MMP-2 expression in the fibroblast (see also FIG. 29).

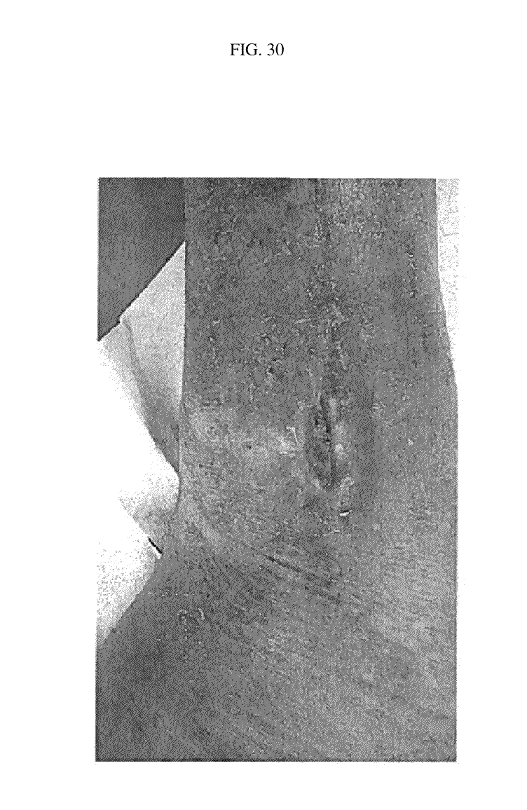

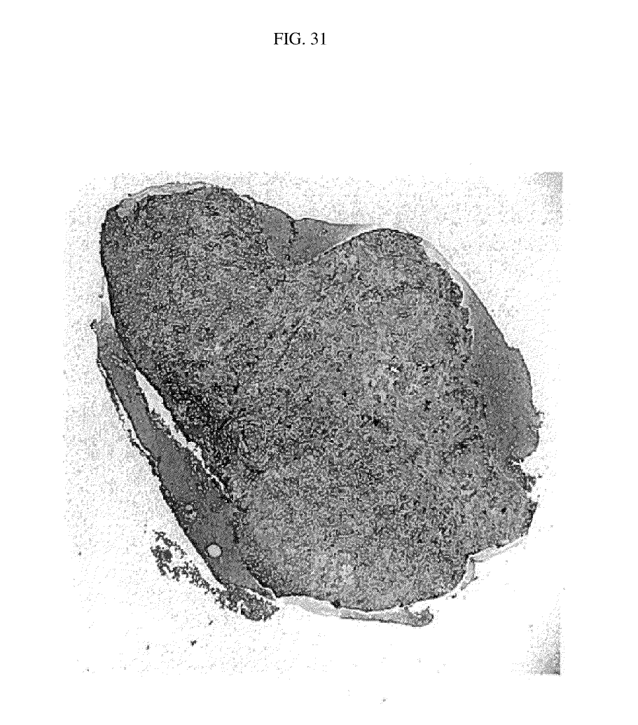

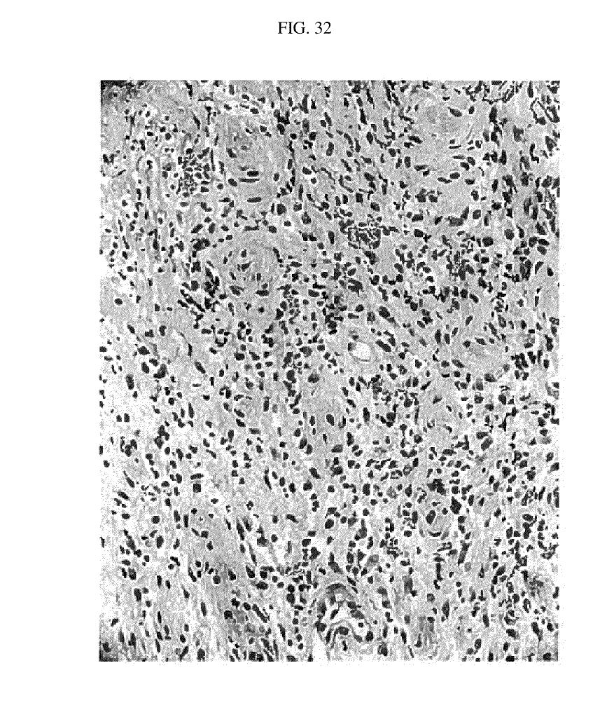

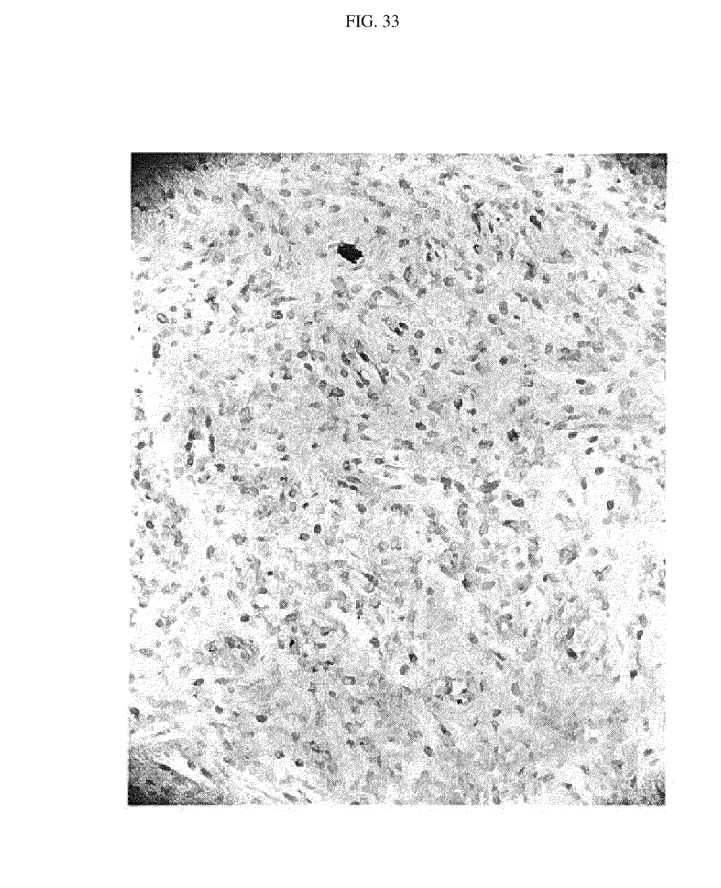

At week 5, the wound was almost fully closed and the fibrinous cap had further diminished (FIGS. 30 and 31). Biopsies of the wound at week 5 showed slightly more active stellate fibroblasts, an increase in inflammation and a decrease of MMP-2 expression. (FIGS. 32 and 33).

As noted, complete healing of the wound occurred after only 7 weeks of treatment.

### Example V

### Female 76 years of age

History
Rheumatoid arthritis
Morbus Reynaud
Lumbal sympatechtomy
Amputation of first digit
Venous insufficiency

Type of Wound
Leg ulcer right medial ulcus
Leg ulcer left medial ulcus

Duration of wound
Wounds had existed for more than 4 years (open/healed)

Earlier Treatment
SSD (FLAMMAZINE)
BIATIN foam,
Compression bandage

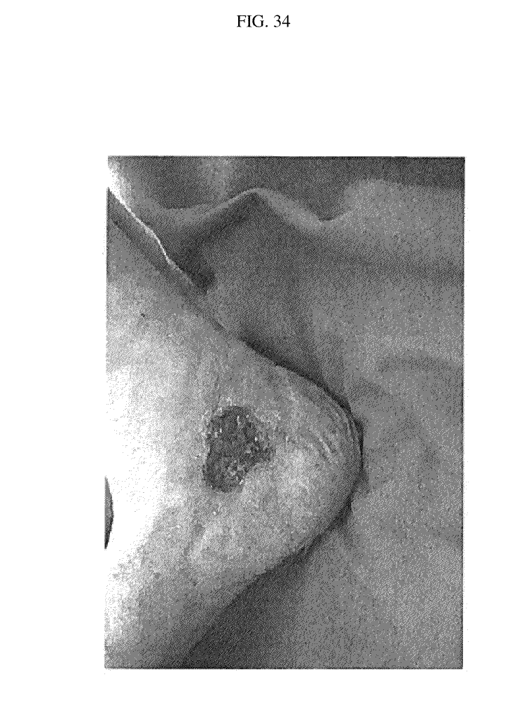

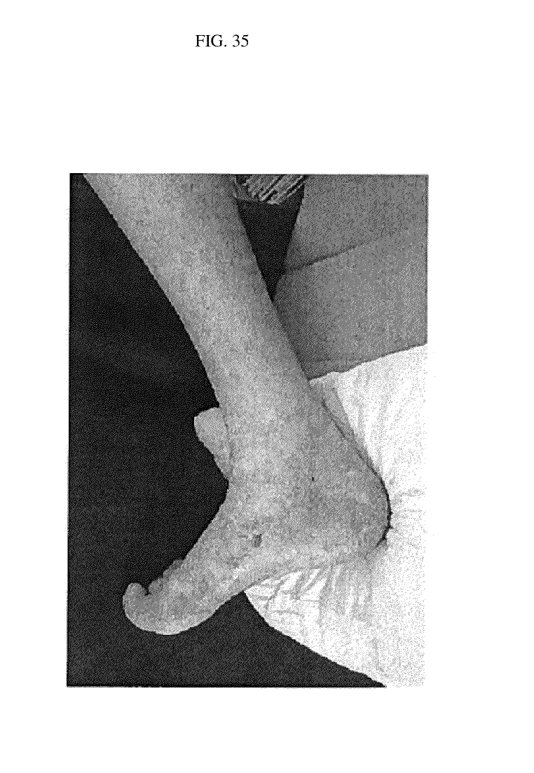

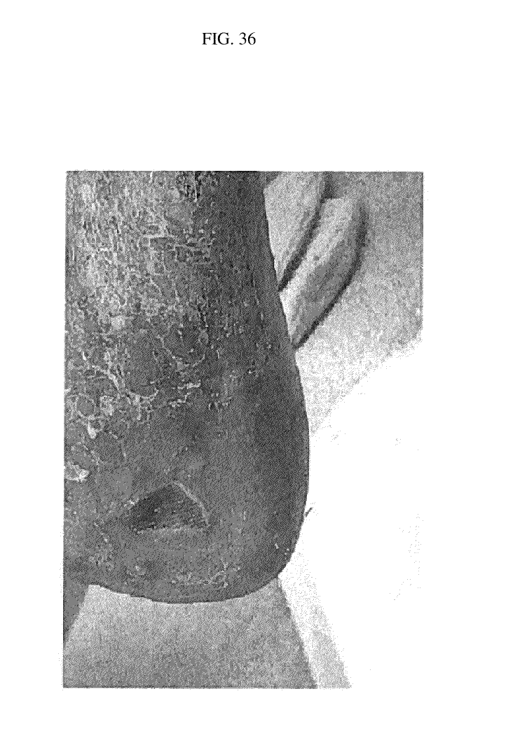

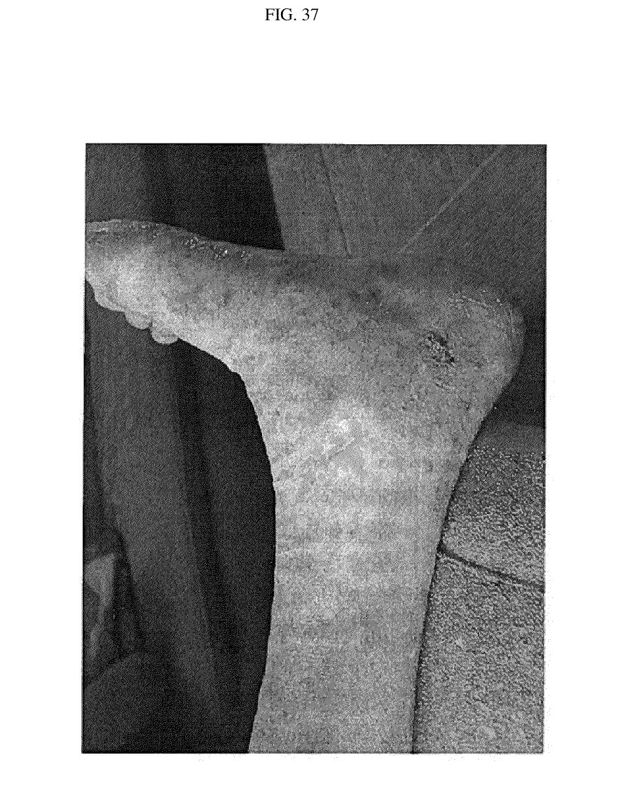

FIGS. 35 and 36 depict the two wounds involved in Example V. FIGS. 35 and 37 depict the same two wounds after fully healed. Full healing of the wound of FIG. 34 was effected after 8½ months of treatment, and full healing of the wound of FIG. 36 was effected after 9 months of treatment.

In one embodiment, the composition of the present invention includes zinc ions, rubidium ions, potassium ions, and calcium ions.

Solutions including various of the above-listed ingredients were prepared as follows:

Composition I

| | | |
|---|---|---|
| potassium citrate | 0.895 | moles/l |
| rubidium chloride | 3.1 | millimoles/l |
| zinc chloride | 64 | micromoles/l |
| citric acid | | (sufficient to adjust |
| | | the pH of the solution |
| | | to 5.5) |

Composition II

| | | |
|---|---|---|
| potassium citrate | 0.895 | moles/l |
| rubidium chloride | 3.1 | millimoles/l |
| zinc chloride | 64 | micromoles/l |
| calcium chloride | 0.2 | millimoles/l |
| citric acid | | (sufficient to adjust |
| | | the pH of the solution |
| | | to 5.5) |

Composition III

| | | |
|---|---|---|
| potassium hydroxide | 0.895 | moles/l |
| rubidium chloride | 3.1 | millimoles/l |
| zinc chloride | 64 | micromoles/l |
| citric acid | | (sufficient to adjust |
| | | the pH of the solution |
| | | to 5.5) |

Composition IV

| | | |
|---|---|---|
| potassium hydroxide | 0.895 | moles/l |
| rubidium chloride | 3.1 | millimoles/l |
| zinc chloride | 64 | micromoles/l |
| calcium chloride | 0.2 | millimoles/l |
| citric acid | | (sufficient to adjust |
| | | the pH of the solution |
| | | to 5.5) |

Preferably, pharmaceutical grade ingredients are employed in each composition of the present invention.

Compositions I and III were subjected to chemiluminescence assay (indicative of inhibition of production of reactive oxygen species, complement assay (classical pathway, indicative of complement activity). These compositions of the present invention exhibited IC-50 values as follows:

TABLE A

| | Chemiluminescence | Complement |
|---|---|---|
| | Assay | Assay |
| Example I | 10 μl/ml | 9 μl/ml |
| Example II | 36 μl/ml | 28 μl/ml |

Composition II which included potassium hydroxide required a greater amount of citric acid to produce a pH of 5.0, indicating that the potassium citrate employed in Example I was more active, hence the lower IC-50 values exhibited by Composition I. In any event the complement assay results clearly show the effectiveness of the present composition in the modulation of MMPs found in chronic wounds such as diabetic ulcers, decubitus ulcers, and other wounds.

In one embodiment, the composition of the present invention may be incorporated into a pharmaceutically acceptable carrier such as WHITFIELD'S ointment or other suitable crème.

A composition of the present invention, preferably in a crème-type carrier, may be applied directly to an open wound or the like or through the use of a gauze bandage to which the composition is applied.

A preferred composition for use in the treatment of various open wounds comprises 0.895 moles/l potassium citrate, 3.1 millimoles rubidium chloride, 0.2 millimoles/l calcium chloride and 64 micromoles zinc chloride in a solution employing distilled, water. The solution is acidified to pH 5.0 employing citric acid.

Whereas the compositions of the present invention function to scavenge superoxide anions, addition of other pharmaceutically acceptable scavengers of superoxide anions may be employed. Naturally occurring polyether or caffeic acid may be beneficial in the treatment of wounds, particularly burn wounds. Such additives may reduce the chemiluminescence and/or DPPH assay (anti-oxidant activity by donating electrons or hydrogen) values of the composition into the microgram range.

The preferred composition of the present invention may be modified by eliminating calcium ions, but with some reduction in the efficacy of the composition, in treating at least certain wounds. As noted, substitution of potassium hydroxide for potassium citrate in the present composition is permissible, but not preferred, due to the increased need for acid to adjust the pH of the solution to 5.0. Though present in a relatively small amount, the presence of zinc ions in the solution appear to be important to the desired level of effectiveness of the present composition. This same factor appears true for rubidium ions. Whereas the sources of the inorganic ions of the present composition are given herein, it is to be recognized that other sources of these ions may be acceptable for given applications of the composition. Initial tests have indicated that the quantity of the several inorganic ions in the composition may be varied from the preferred composition without destruction of, but with possible reduction of, the wound healing efficacy of the composition. In all instances, preferably, the pH of the solution is adjusted to substantially 5.0 thereby imparting desirable buffering properties to the composition.

In any event, the Active ingredients of the present composition have been found to include zinc, potassium, rubidium and/or calcium. Calcium does not appear to be critical to the desired healing process, it does not appear to be detrimental when included in the present composition, and in certain instances is considered desirable. On the other hand, zinc appears to be essential to the healing qualities of the present composition, and rubidium is also strongly indicated for those compositions employed in cancer, ulcer and others of those maladies for which the present compositions have been found useful as healing agents.

Citric acid, preferably, when included in the present composition for pH control purposes has been found effective in such role. Other acids for normalizing the pH of present solution, for example hydrochloric acid, may be employed.

Polyethylene glycol has been found particularly effective as a component of the present solution, in part due to its oxygen scavenging properties.

In one embodiment of the present invention, a channeling agent, such as monoxidil, has been found to be effective in lieu of the potassium ions.

Whereas the compositions of the present invention may include other inactive ingredients which are biologically relatively inert or inactive relative to the healing process of a wound, the present inventors have found that the absence of ions of zinc, potassium, rubidium and calcium (in certain compositions) are essential to obtaining the aforenoted dramatic results of wound healing.

During wound repair, different MMPs are produced by multiple cell types. MMP-2 is produced only by inflammatory cells. MMP-9 is produced by keratinocytes as well as inflammatory cells. MMP-2 and MMP-9 act on cleaved collagen better than other MMPs. MMPs are not actively expressed in uninjured skin either in the epidermis or dermis. The idea exists that MMPs are stored in the matrix awaiting activation by migrating cells. Inflamed tissues in chronic wounds exhibit excessively high MMP levels in comparison to normal healing wounds, the excess being in the range of 30% greater MMP levels in chronic wounds.

In accordance with one aspect of the present invention, the compositions of the present invention exhibit those properties which are known to increase tissue regeneration of chronic open wounds, providing full wound closure of demonstrated non-responding or slow-healing wounds.

At a first level, compositions of the present invention clearly modulate the expression of one or more MMPs, particularly MMP-2 and MMP-9, thereby reducing the levels of these MMPs in chronic wounds to normalize wound healing. At second and further levels, compositions of the present invention function to scavenge oxygen radicals from wound sites, normalizing the pH levels within a wound and thereby developing an environment within the wound which is favorable to healing. Still further, the compositions also can reduce inflammation, scavenge tree oxygen radicals, reduce scar tissue, and act as a powerful antimicrobal.

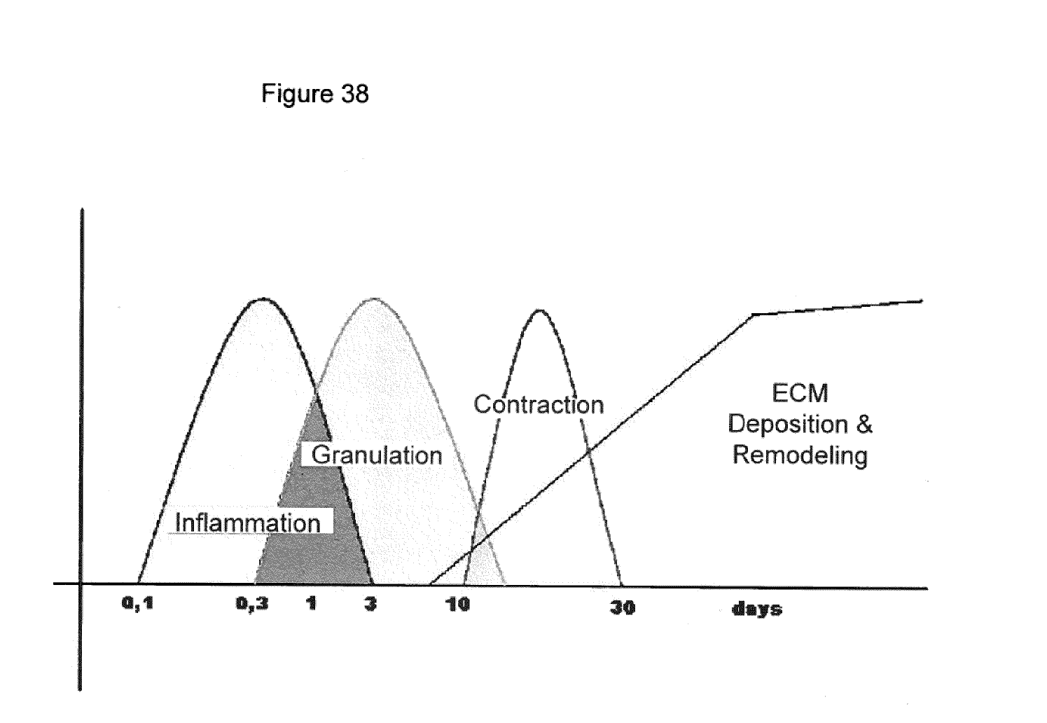

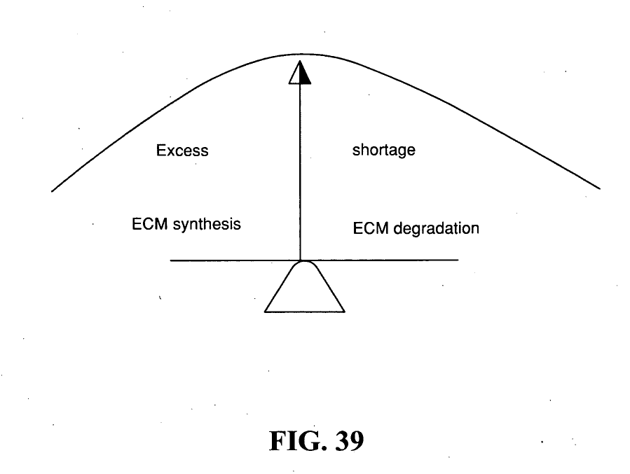

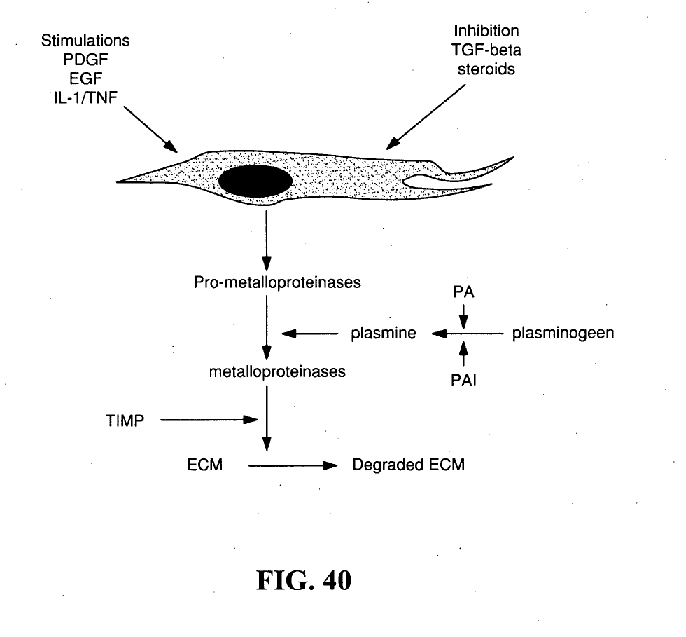

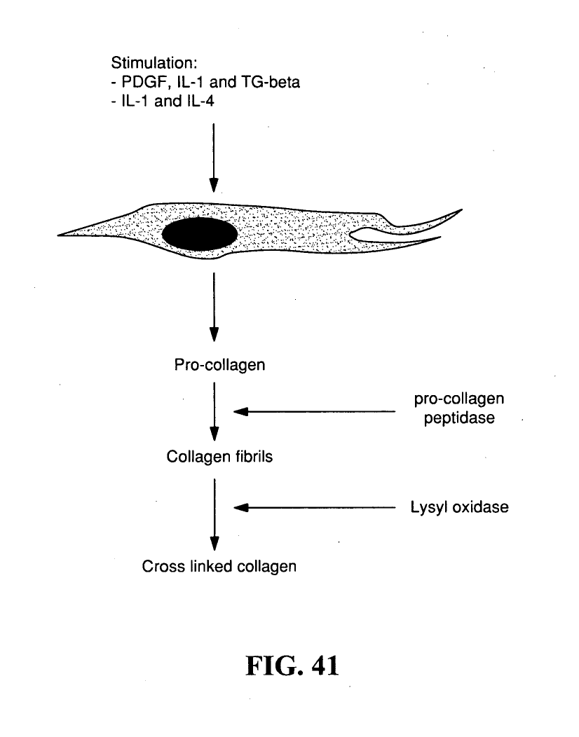

Dermal wound healing is recognized as a complex, but orderly process which takes place in injured tissue. Subsequently the injured tissue respond with inflammation, granulation tissue formation, extracellular matrix (ECM) deposition, contraction and remodeling of the deposited collagen. This process is depicted in FIG. 37. The present inventors have found that remodeling results when there is a balance between ECM-synthesis and ECM-degradation. Many different circumstances can influence these processes thus shifting the balance toward a state of excess or shortage of ECM, thereby inhibiting the remodeling process (See FIG. 38). As seen in FIG. 39, fibroblast synthesis of collagen, the major constituent of the dermal tissue, is stimulated by growth factors and cytokines. Soluble pro-collagen peptides are released in the environment of the fibroblasts. Procollagen peptidase cleaves of the terminal peptide chains allow true collagen fibrils to form. Lysyl-oxidase promotes the cross-linking of these fibrils rendering structural stability to the matrix. In the ECM, several types of collagen can be recognized, along with other substances which contribute to the ECM.

The production of MMPs, enzymes that serve to degrade collagen, are also under the influence of growth factors. Stimulating and inhibition factors result I the release of pro-metalloproteinases. These pro-forms are activated by plasmine. Activated metalloproteinases are quickly deactivated by Tissue Inhibitors of metalloproteinases (TIMPs) so that the spatial actin of the proteolytic enzyme is limited. The main action of the MMPs is to degrade the collagen. It has to be borne in mind that this scheme is likely to be an oversimplification of what is happening in vivo. For example, (a) plasmine release from plasminogeen is regulated by the action of plasminogeen activator (PA) and plasminogeen Activator Inhibitor (PAI) both of which are also produced by fibroblasts under the influence of growth factors and cytokines; (b) Metalloproteinases can also be activated by other substances as HOCL— from the oxidative burst of granulocytes (H₂ O₂+MPO Cl⁻→HOCl— which is strongly anti-bacterial); (c) metalloproteinases can also be activated by other than TIMP, for instance alpha2-Macroglobulin (anti-protease in serum); and/or (d) metalloproteinases can cleave other molecules than collagen for instance other ECM molecules by cleavage capacity can perhaps also lead to activation of the complement system (C5a and C3A).

Very little appears to be known about the distribution of MMPs in time. It is known that normal skin shows basic levels of MMP-2, but shows no MMP-9 expression. The present inventors have shown elevated levels of MMPs in chronic wounds.

Irrespective of the complexity of the wound healing mechanism, the present inventors have discovered a combination of metal ions which in solution, preferably substantially at a pH of 5.0, when applied over time to a chronic or other dermal wound, dramatically enhances the healing of the wound. The composition of the present invention is further indicated in the treatment of cancers, psoriasis, and a variety of skin infections, burns, and/or lesions.

What is claimed:

1. A method for therapeutic modulation of one or more matrix metalloproteinases (MMPs), comprising the steps of:
identifying an elevated level of one or more MMPs expressed in tissue;
administering a composition to the tissue to modulate the expression of one or more MMPs, wherein said composition comprises a pharmaceutically effective amount of a combination of zinc and rubidium ions in a physiologically inert carrier;
monitoring the demodulation of the one or more MMPs in the tissue; and
re-administering the composition to the tissue until the one or more MMP levels return near zero.

2. The method of claim 1, wherein the tissue being therapeutically modulated for MMP expression is a chronic wound.

3. The method of claim 2, wherein the chronic wound being treated is an open, full thickness tissue wound.

4. The method of claim 2, wherein the chronic wound being treated is subsurface traumatized tissue.

5. The method of claim 4, wherein the identifying step further comprises taking and examining a biopsy of the tissue to determine whether an elevated level of the one or more MMPs is being expressed.

6. The method of claim 5, wherein the monitoring step further comprises taking and examining a biopsy of the tissue to determine whether the expression of the one or more MMPs has declined.

7. The method of claim 6, wherein the administering and re-administering steps further comprise substantially and fully filling a wound cavity with the composition to ensure that the composition reaches both the outer edges of the wound cavity and the deep, inner recesses of the wound cavity.

8. The method of claim 4, wherein the composition applied in the administering step further comprises a pharmaceutically effective amount of calcium ions.

9. The method of claim 4, wherein the composition applied in the administering step further comprises a pharmaceutically effective amount of potassium ions.

10. The method of claim 4, wherein the composition applied in the administering step further comprises a sufficient amount of an acid to adjust the pH to about 5.0

11. The method of claim 10, wherein the composition applied in the administering step further comprises a sufficient amount of citric acid to adjust the pH to about 5.0.

12. A method for therapeutic modulation of a matrix metalloproteinase (MMP), comprising the steps of:
identifying an elevated level of MMP-2 or MMP-9 expressed in tissue;
administering a composition to the tissue to modulate the expression of MMP-2 or MMP-9, wherein said composition comprises a pharmaceutically effective amount of a combination of zinc and rubidium ions in a physiologically inert carrier;
monitoring the demodulation of the MMP-2 or MMP-9 in the tissue; and
re-administering the composition to the tissue until MMP levels return near zero.

13. The method of claim 12, wherein the tissue being therapeutically modulated for MMP expression is a chronic wound.

14. The method of claim 12, wherein the identifying step further comprises taking and examining a biopsy of the tissue to determine whether an elevated level of MMP-2 or MMP-9 is being expressed.

15. The method of claim 14, wherein the monitoring step further comprises taking and examining a biopsy of the tissue to determine whether the expression of MMP-2 or MMP-9 has declined.
